(19) United States
(12) Patent Application Publication
(10) Pub. No.: US 2016/0263094 A1
(43) Pub. Date: Sep. 15, 2016

(54) Pantoprazole Sodium Composition Lyophilized Powder for Injection

(71) Applicant: HAINAN WEI-KANG PHARMACEUTICAL (QIANSHAN) COMPANY LIMITED, Anqing City. Anhui (CN)

(72) Inventors: Liuyi Wang, Hefei City, Anhui (CN); Jincan Wang, Anqing City, Anhui (CN); Dingwen Zhou, Anqing City, Anhui (CN)

(21) Appl. No.: 14/411,848

(22) PCT Filed: Jun. 11, 2014
(86) PCT No.: PCT/CN2014/079649

§ 371 Date: Dec. 29, 2014

(30) Foreign Application Priority Data
Oct. 15, 2013 (CN) 201310481775.0

(51) Int. Cl. A61K 31/4439; A61K 47/36; A61K 9/00; A61K 9/19

(52) U.S. Cl. CPC A61K 31/4439; A61K 9/19; A61K 47/36; A61K 9/0019

(57) ABSTRACT

The invention provides pantoprazole sodium composition lyophilized powder for injection, and relates to the technical field of medicines and manufacturing of the medicines. The pantoprazole sodium composition lyophilized powder for injection comprises the following raw material ingredients in parts by weight: 7.24-9.08 parts of pantoprazole sodium, 4.34-5.45 parts of chitosan nanoparticles and 85.25-89.11 parts of water for injection. The invention has the following advantages: 1) a composition comprising pantoprazole sodium and chitosan nanoparticles according to a ratio of 1:0.6 has a stronger effect of inhibiting an H+-K+ATPase; 2) the using amount of pantoprazole sodium can be clinically reduced, the adverse reactions of pantoprazole sodium are reduced, and 20 mg of pantoprazole sodium containing the chitosan nanoparticles has the same effect of inhibiting secretion of gastric acid as 30 mg of pantoprazole sodium without the chitosan nanoparticles; 3) the water solubility of the medicament is improved, and the good stability of the medicament is simultaneously ensured; 4) the medicament-carrying nanoparticles can change a membrane operation mechanism, increase the permeability of the medicament across a biological membrane and be conductive to realize an efficacy of the medicament in cells; and 5) the chitosan nanoparticles can replace mannitol to serve as a lyophilized skeleton agent of the lyophilized powder, thereby eliminating an active effect of mannitol on a human body.

### FIELD OF THE INVENTION

The invention relates to the technical field of medicines and manufacturing of the medicines and particularly relates to pantoprazole sodium composition lyophilized powder for injection.

### BACKGROUND OF THE INVENTION

The chemical name of pantoprazole sodium is 5-difluoromethoxy-2-[(3, 4-dimethoxy-2-pyridinyl) methyl]sulfinyl-1H-benzimidazole. The structural formula is as follows:

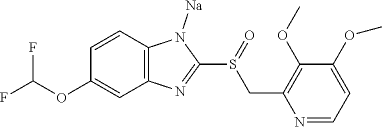

The product is a gastric parietal cell proton pump inhibitor and can specifically inhibit secretory microtubules constituted by membranes at the top ends of parietal cells and an H+/K+-ATPase on a tubular bulb in cytoplasm, cause irreversible inhibition of the enzyme and further effectively inhibit secretion of gastric acid. As the H+/K+-ATPase is the last process of acid secretion of the parietal cells, the product has strong acid inhibition capability. The product can not only realize non-competitive inhibition of secretion of gastric acid caused by gastrin, histamine and choline, but also inhibit part of basic secretion of gastric acid, which is not affected by choline or an H2 receptor antagonist.

Pantoprazole sodium is clinically widely used for treating gastric and duodenal ulcers and reflux esophagitis. Due to a significant curative effect and relatively low recurrence rate, as soon as pantoprazole sodium appeared on the market, pantoprazole sodium became a first-line medicament for treating abnormal secretion of gastric acid and related diseases. However, with the wide clinical application of PPIs, reports about adverse reactions and interactions of the medicament are increasing year by year.

Chitosan is a product obtained by removal of N-acetyl from chitin, the structure is very similar to that of cellulose, except a hydroxyl group is replaced by an acetyl amino group on C2 of a saccharide ring, and the acetyl amino group endues chitosan with special properties, so that chitosan can be used in the aspect of pharmaceutical preparations. Many physiological activities of chitosan enable chitosan to be widely applied in the field of medicines.

Pantoprazole sodium acts on the key enzyme, namely the H+/K+-ATPase in the final step of the secretion of gastric acid of the parietal cells; and chitosan nanoparticles have an effect of adsorbing the enzyme, can finally realize irreversible inactivation of the enzyme, enable the acid inhibition effect to be stronger and last longer, realize high-speed promotion of healing of ulcers and high healing rate, and are suitable for treatment of various refractory ulcers or treatment for patients with NSAID ulcers who can not stop the use of NSAID. However, at present, there is still no pharmaceutical formulation containing the chitosan nanoparticles clinically.

### SUMMARY OF THE INVENTION

One purpose of the invention is to provide a pantoprazole sodium lyophilized powder composition for injection. The composition comprises main ingredients of pantoprazole sodium and chitosan nanoparticles.

The technical problem to be solved by the invention is implemented by adopting the following technical scheme.

A prescription of the pantoprazole sodium composition provided by the invention comprises pantoprazole sodium, chitosan nanoparticles and water for injection, and the pantoprazole sodium composition is characterized in that the chitosan nanoparticles can be used as a skeleton agent, a solubilizer and a synergist of the pantoprazole sodium (the chitosan nanoparticles have a certain activity of inhibiting secretion of gastric acid and can realize a synergistic effect after being combined with the pantoprazole sodium).

The invention provides pantoprazole sodium composition lyophilized powder for injection, characterized by comprising the following raw material ingredients in parts by weight:

| | |
|---|---|
| pantoprazole sodium | 7.24-9.08 parts |
| chitosan nanoparticles | 4.34-5.45 parts |
| water for injection | 85.25-89.11 parts |

The invention further provides a preparation method of the pantoprazole sodium composition lyophilized powder for injection, which is characterized by comprising the following steps:

(I) preparation of chitosan nanoparticles:
1) crushing chitosan powder and then screening with a 100-mesh screen;
2) weighing 100 g of chitosan powder, adding into 40 L of 0.1 mol/l acetic acid solution at room temperature (20° C.), and performing magnetic stirring to completely dissolve chitosan, thus obtaining a chitosan acetic acid solution (C=2.5 g/L);
3) regulating pH to 5.0 with 1% NaOH;
4) adding 1667 g of 1% sodium tripolyphosphate into the chitosan acetic acid solution while stirring, and ensuring that the mass ratio of chitosan/sodium tripolyphosphate is 6:1, thus obtaining the nanoparticles through crosslinking under an electrostatic effect of anions and cations; and
5) performing high-speed centrifugation (18000 r/min) on the colloidal solution at the temperature of 4° C. for 30 min, collecting a lower-layer precipitate, washing with pure water three times, then cooling, and further performing vacuum drying (below 30° C.) to obtain the chitosan nanoparticles, wherein the moisture content is lower than 2%, the particle size is not more than 100 nm, and the zeta potential is about 15 mv;
(II) preparation of the pantoprazole sodium composition lyophilized powder for injection:
1) slowly adding the prescribed amount of chitosan nanoparticles into the prescribed amount of water for injection while stirring till dissolution;
2) adding the prescribed amount of pantoprazole sodium, stirring, and dissolving till clarification;
3) regulating pH to 5.1 with buffer salts of sodium dihydrogen phosphate and disodium hydrogen phosphate, adding 0.1% activated carbon, stirring for 30 min, filtering out the activated carbon, filtering medicine liquid through 0.45 μm and 0.22 μm microporous filter membranes, detecting the content of an intermediate, and calculating the filling amount according to 20 mg of pantoprazole sodium per bottle; and
4) filling according to detection requirements, performing semi-plugging, then sending into a lyophilizing machine, reducing the temperature to −40° C., keeping the temperature for 2 h, slowly increasing the temperature to −5° C.-0° C., sublimating, drying, further increasing the temperature to 35° C., keeping the temperature for 3 h, and taking out of a box after the end of lyophilizing.

The invention has the following beneficial effects:

the invention provides a composition formed by mixing pantoprazole sodium and the chitosan nanoparticles according to a ratio of 1:0.6 and prepares a lyophilized powder for injection from the composition to serve as a clinical medicament for treating gastric and duodenal ulcers. Multiple experiments made by the inventors prove that the composition has the following advantages: 1) the composition comprising the pantoprazole sodium and the chitosan nanoparticles according to the ratio of 1:0.6 has a stronger effect of inhibiting an H+-K+ATPase; 2) in-vitro experiments prove that the using amount of pantoprazole sodium can be clinically reduced, the adverse reactions of pantoprazole sodium are reduced, and 20 mg of pantoprazole sodium containing the chitosan nanoparticles has the same effect of inhibiting secretion of gastric acid as 30 mg of pantoprazole sodium without the chitosan nanoparticles; 3) the water solubility of the medicament is improved, and the good stability of the medicament is simultaneously ensured; 4) the medicament-carrying nanoparticles can change a membrane operation mechanism, increase the permeability of the medicament across a biological membrane and be conductive to realize an efficacy of the medicament in cells; and 5) the chitosan nanoparticles can replace mannitol to serve as a lyophilized skeleton agent of the lyophilized powder, thereby eliminating an active effect of mannitol on a human body.

### DETAILED DESCRIPTION OF THE INVENTION

The following embodiments are used for describing the invention rather than limiting the scope of the invention.

### Embodiment 1

### Preparation of Pantoprazole Sodium Composition Lyophilized Powder for Injection, Measured by 1000 Pieces

### 1. Prescription:

| | | |
|---|---|---|
| pantoprazole sodium | 20 | g |
| chitosan nanoparticles | 12 | g |
| water for injection | 2000 | ml |

### 2. Preparation Process:

Weighing 12 g of chitosan nanoparticles, and slowly adding into 2000 ml of water for injection while stirring till dissolution.

Adding 20 g of pantoprazole sodium, stirring, and dissolving till clarification.

Regulating PH to 5.1 with buffer salts of sodium dihydrogen phosphate and disodium hydrogen phosphate, adding 0.1% activated carbon, stirring for 30 min, filtering out the activated carbon, filtering medicine liquid through 0.45 μm and 0.22 μm microporous filter membranes, detecting the content of an intermediate, and calculating the filling amount according to 20 mg of pantoprazole sodium per bottle.

Filling according to detection requirements, performing semi-plugging, then sending into a lyophilizing machine, reducing the temperature to −40° C., keeping the temperature for 2 h, slowly increasing the temperature to −5° C.-0° C., sublimating, drying, further increasing the temperature to 35° C., keeping the temperature for 3 h, and taking out of a box after the end of lyophilizing.

### Embodiment 2

### Preparation of Pantoprazole Sodium Composition Lyophilized Powder for Injection, Measured by 1000 Pieces

### 1. Prescription:

| | | |
|---|---|---|
| pantoprazole sodium | 20 | g |
| chitosan nanoparticles | 8 | g |
| water for injection | 2000 | ml |

### 2. Preparation Process:

Weighing 8 g of chitosan nanoparticles, and slowly adding into 2000 ml of water for injection while stirring till dissolution.

Adding 20 g of pantoprazole sodium, stirring, and dissolving till clarification.

Regulating PH to 5.1 with buffer salts of sodium dihydrogen phosphate and disodium hydrogen phosphate, adding 0.1% activated carbon, stirring for 30 min, filtering out the activated carbon, filtering medicine liquid through 0.45 μm and 0.22 μm microporous filter membranes, detecting the content of an intermediate, and calculating the filling amount according to 20 mg of pantoprazole sodium per bottle.

Filling according to detection requirements, performing semi-plugging, then sending into a lyophilizing machine, reducing the temperature to −40° C., keeping the temperature for 2 h, slowly increasing the temperature to −5° C.-0° C., sublimating, drying, further increasing the temperature to 35° C., keeping the temperature for 3 h, and taking out of a box after the end of lyophilizing.

### Embodiment 3

### Preparation of Pantoprazole Sodium Composition Lyophilized Powder for Injection, Measured by 1000 Pieces

### 1. Prescription:

| | | |
|---|---|---|
| pantoprazole sodium | 20 | g |
| chitosan nanoparticles | 16 | g |
| water for injection | 2000 | ml |

### 2. Preparation Process:

Weighing 16 g of chitosan nanoparticles, and slowly adding into 2000 ml of water for injection while stirring till dissolution.

Adding 20 g of pantoprazole sodium, stirring, and dissolving till clarification.

Regulating PH to 5.1 with buffer salts of sodium dihydrogen phosphate and disodium hydrogen phosphate, adding 0.1% activated carbon, stirring for 30 min, filtering out the activated carbon, filtering medicine liquid through 0.45 μm and 0.22 μm microporous filter membranes, detecting the content of an intermediate, and calculating the filling amount according to 20 mg of pantoprazole sodium per bottle.

Filling according to detection requirements, performing semi-plugging, then sending into a lyophilizing machine, reducing the temperature to −40° C., keeping the temperature for 2 h, slowly increasing the temperature to −5° C.-0° C., sublimating, drying, further increasing the temperature to 35° C., keeping the temperature for 3 h, and taking out of a box after the end of lyophilizing.

### Experimental Data

50 SD rats are used, made to starve for 24 h before testing. The abdominal cavity is opened under ether anesthesia, a glass tube with the inner diameter of 5 mm and the length of 30 mm is vertically placed on the surface of serosa of the gastric body and 0.2 ml of glacial acetic acid is added into the tube cavity.

The glacial acetic acid is dipped out with a cotton swab after 5 min, and the surgical incision is sutured. Normal diet is permitted after the surgery, and animals are randomly divided into 5 groups on the next day: a control group 1 (distilled water), a control group 2 (pantoprazole sodium for injection), with continuous administration for 15 d, a treatment group 1 (embodiment 1), a treatment group 2 (embodiment 2) and a treatment group 3 (embodiment 3); and continuous administration is performed for 10 d, three times a day. The stomach is taken out by anatomy, fixed with formaldehyde, cut open along the greater curvature of the stomach and flat spread on a glass plate, and the transverse diameter d1 and the vertical diameter d2 of an ulcer are measured. The area of the ulcer S (mm2) is calculated according to a formula S=π×(d1/2)×(d2/2) to serve as an ulcer index, and ulcer healing rate and ulcer inhibition rate are calculated. The results are shown in the following table.

| | | Number | | | |
|---|---|---|---|---|---|
| | | of | Healing | | Ulcer |
| | | healed | rate | Ulcer index | inhibition |
| Group | Dose (mg/kg) | animals | (%) | x ± s, mm2) | rate (%) |
| Control | | 0 | 0 | 19.3 ± 8.7 | |
| group 1 | | | | | |
| Control | 1.3 | 9 | 90 | 6.3 ± 6.1 | 65.1 |
| group 2 | (pantoprazole) | | | | |
| Treatment | 1.3 | 10 | 100 | 2.5 ± 3.3 | 84.6 |
| group 1 | (pantoprazole | | | | |
| | composition) | | | | |
| Treatment | 1.3 | 10 | 100 | 2.6 ± 3.4 | 86.2 |
| group 2 | (pantoprazole | | | | |
| | composition) | | | | |
| Treatment | 1.3 | 9 | 90 | 2.8 ± 3.8 | 85.4 |
| group 3 | (pantoprazole | | | | |
| | composition) | | | | |

The experimental data shows that embodiments 1, 2 and 3 have a good effect of treating ulcers, the healing rate of each of the treatment groups 1, 2 and 3 is higher than that of the group using pantoprazole sodium alone (control group 2), the effect of treating a gastric ulcer is good and the ulcer area can be repaired. The treatment time of each of the treatment groups 1, 2 and 3 is less than that of the group using pantoprazole sodium alone (control group 2) by ⅓, and the dose is less by 30%.

The above contents illustrate and describe the basic principle, the main characteristics and the advantages of the invention. Those of skill in the industry should understand that the invention is not limited by the above embodiments. The above embodiments and the descriptions in the specification are only preferred embodiments of the invention and are not used for limiting the invention. Without deviating from the spirit and scope of the invention, various changes and improvements can be made to the invention, and these changes and improvements fall within the protection scope of the invention. The protection scope of the invention is defined by the appended claims and equivalents thereof.

1. Pantoprazole sodium composition lyophilized powder for injection, characterized by comprising the following raw material ingredients in parts by weight:

| | |
|---|---|
| pantoprazole sodium | 7.24-9.08 parts |
| chitosan nanoparticles | 4.34-5.45 parts |
| water for injection | 85.25-89.11 parts |

2. A preparation method of the pantoprazole sodium composition lyophilized powder for injection according to claim 1, characterized by comprising the following steps:
(I) preparation of chitosan nanoparticles:
1) crushing chitosan powder and then screening with a 100-mesh screen;
2) weighing 100 g of chitosan powder, adding into 40 L of 0.1 mol/l acetic acid solution at room temperature, and performing magnetic stirring to completely dissolve chitosan, thus obtaining a chitosan acetic acid solution;
3) regulating pH to 5.0 with 1% NaOH;
4) adding 1667 g of 1% sodium tripolyphosphate into the chitosan acetic acid solution while stirring, and ensuring that the mass ratio of chitosan/sodium tripolyphosphate is 6:1, thus obtaining the nanoparticles through crosslinking under an electrostatic effect of anions and cations; and
5) performing high-speed centrifugation on the colloidal solution at the temperature of 4° C. for 30 min, collecting a lower-layer precipitate, washing with pure water three times, then cooling, and further performing vacuum drying to obtain the chitosan nanoparticles, wherein the moisture content is lower than 2%, the particle size is not more than 100 nm, and the zeta potential is about 15 mv;
(II) preparation of the pantoprazole sodium composition lyophilized powder for injection:
1) slowly adding the prescribed amount of chitosan nanoparticles into the prescribed amount of water for injection while stirring till dissolution;
2) adding the prescribed amount of pantoprazole sodium, stirring, and dissolving till clarification;
3) regulating pH to 5.1 with buffer salts of sodium dihydrogen phosphate and disodium hydrogen phosphate, adding 0.1% activated carbon, stirring for 30 min, filtering out the activated carbon, filtering medicine liquid through 0.45 μm and 0.22 μm microporous filter membranes, detecting the content of an intermediate, and calculating the filling amount according to 20 mg of pantoprazole sodium per bottle; and
4) filling according to detection requirements, performing semi-plugging, then sending into a lyophilizing machine, reducing the temperature to −40° C., keeping the temperature for 2 h, slowly increasing the temperature to −5° C.-0° C., sublimating, drying, further increasing the temperature to 35° C., keeping the temperature for 3 h, and taking out of a box after the end of lyophilizing.
